(19) United States
(12) Patent Application Publication
(10) Pub. No.: US 2007/0026028 A1
(43) Pub. Date: Feb. 1, 2007

(54) Appliance for delivering a composition

(76) Inventors: Kenneth B. Close, New London, WI (US); Wael R. Joseph, Appleton, WI (US); Gary V. Anderson, Larsen, WI (US); Jonathan K. Arendt, Appleton, WI (US); Gregory K. Hall, Menasha, WI (US); Kroy D. Johnson, Menasha, WI (US); Laura E. Keck, Alpharetta, GA (US); MeeWha Lee, Appleton, WI (US)

Correspondence Address: KIMBERLY-CLARK WORLDWIDE, INC., 401 NORTH LAKE STREET, NEENAH, WI, 54956, US

(21) Appl. No.: 11/190,597

(22) Filed: Jul. 26, 2005

(51) Int. Cl. A01N 25/34; A61K 8/02; B32B 27/12; B32B 27/32; B32B 5/26; A41D 19/00; B32B 5/02; B32B 27/04; A61K 6/00

(52) U.S. Cl. 424/402; 442/123; 442/382; 442/394; 442/398; 424/401; 2/167

(57) ABSTRACT

Disclosed is an appliance for treating skin or tissue, the appliance comprising a three-layer substrate having a cloth-like appearance and feel. The substrate includes a water-impermeable layer, such as a film, sandwiched between, and attached to, two fibrous layers. The fibrous layer that is adapted to contact skin includes a formulation or composition adapted to be transferred to skin or tissue of the user. Furthermore, the fibrous layer that is adapted to contact skin includes undulations that inhibit leakage of the formulation or composition.

### BACKGROUND

People rely on various kinds of formulations or compositions for health and/or hygiene benefits.

Generally, two categories of formulations are used when moisturizing and/or hydrating skin. Humectants are used to transport moisture from the environment (primarily water vapor in air) to skin. One example of a humectant is glycerin. Such materials tend to be hydrophilic (i.e., water loving), and are generally non-greasy, light in weight and/or viscosity, and quick to apply. Occlusive materials are used to trap moisture already present in the skin. These materials tend to create a water barrier over the skin, thereby inhibiting the transport of water from the skin to the external environment. An example of an occlusive material is petrolatum. Such materials tend to be heavier, effective over longer periods of time, and often contain oily and/or greasy ingredients.

Often people apply such formulations directly to skin using their hands. If the formulation includes water as an ingredient, water may evaporate, potentially decreasing the effectiveness of the formulation. Furthermore, after application of the formulation to the body, any excess formulation remaining on the hands must be removed.

In some instances, gloves or socks have been used in conjunction with formulations. A user either applies a formulation to his or her hand or foot, and then dons or applies a glove over the treated hand or a sock over the treated foot. Alternatively, a user slips on a glove or sock that has been pre-treated with a skin-care formulation. Unfortunately, such items have typically been made of a polymeric material (e.g., neoprene rubber) lacking a cloth-like appearance and feel. Often such items do not conform readily to the complex surfaces and contours of a foot or hand. Furthermore, such items may not incorporate an undulating surface on the interior of the glove or sock (i.e., a rugose material), in part to inhibit leakage of the formulation.

What is needed is an appliance that promotes health and/or hygiene by facilitating transport of a formulation or composition to skin or tissue, and which: has a cloth-like appearance and feel; conforms readily to the contours and surfaces of parts of the body to which the appliance is applied; and reduces leakage of the formulation or composition coated or deposited on the interior surface of the appliance.

### SUMMARY

We have determined that an appliance, such as a sleeve, sock, or glove, that comprises a 3-layer substrate (i.e., a water-impermeable layer, such as a film, sandwiched between, and attached to, two fibrous substrates, such as nonwoven materials), and to which a composition or formulation has been deposited for transference to a user's skin or tissue, effectively and comfortably treats the skin or tissue of a user. Preferably the composition or formulation is water-based and includes a humectant, in effect giving the appliance the benefit of both a humectant (providing water molecules for transport into the skin) and an occlusive material (the water-impermeable layer of the substrate reduces or inhibits the transport of water molecules away from the skin or formulation). But materials of an occlusive nature may be used, and numerous other ingredients may be included in the formulation, examples of which are provided below in the Description section. The outer fibrous substrate has a cloth-like appearance and feel, and the rugose nature of the inner fibrous substrate helps reduce leakage of the composition or formulation. The rugosity of the inner fibrous layer, the outer fibrous layer, or both may be imparted or increased by thermally point bonding a fibrous layer (or layers) to the water-impermeable layer while the water-impermeable layer is in a stretched condition. When the resulting laminate is allowed to retract, undulations in one or both fibrous layers are created and/or become more pronounced. The layers may be bonded at discrete locations using an approach other than thermal point bonding (e.g., adhesive bonding). In the Examples section, we detail various representative embodiments of such appliances, along with experimental results that help characterize the benefit of using such appliances.

These and other versions of the invention are described more fully below.

### DRAWINGS

FIG. 1 representatively illustrates one version of a substrate of the present invention.

FIG. 2 representatively illustrates one version of a substrate cut so that the substrate's perimeter defines the shape of a hand.

FIG. 2A representatively illustrates one version of an appliance of the present invention.

FIG. 3 representatively illustrates one version of a substrate cut so as to form a foot appliance of the present invention.

FIG. 3A representatively illustrates one version of an appliance of the present invention.

### DEFINITIONS

Within the context of this specification, each term or phrase below includes the following meaning or meanings:

“Attach” and its derivatives refer to the joining, adhering, connecting, bonding, sewing together, depositing on, associating with, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a foraminous belt. The forming surface may include a gas-pervious material that has been placed onto the forming surface.

“Composition,” “formulation,” or their derivatives, when used in the context of a material applied or deposited on the interior surface of the inner fibrous layer of an appliance of the present invention, refers to the various materials that help improve the health and/or hygiene of a user of the appliance, primarily by helping improve the health and/or hygiene of skin or tissue to which the material is transferred from the interior surface of the inner fibrous layer of the appliance.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Fiber” refers to a continuous or discontinuous member having a high ratio of length to diameter or width. Thus, a fiber may be a filament, a thread, a strand, a yarn, or any other member or combination of these members.

“Hydrophilic” describes materials or surfaces which are wetted by aqueous liquids in contact with the material or surface. The degree of wetting of the material or surface can, in turn, be described in terms of the contact angles and the surface tensions of the liquids and materials (or surfaces) involved.

“Layer” when used in the singular can have the dual meaning of a single element or a plurality of elements.

“Liquid impermeable,” when used in describing a layer or multi-layer laminate means that liquid, such as water, will not pass to any appreciable extent through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblowing processes can be used to make fibers of various dimensions, including macrofibers (with average diameters from about 40 to about 100 microns), textile-type fibers (with average diameters between about 10 and 40 microns), and microfibers (with average diameters less than about 10 microns). Meltblowing processes are particularly suited to making microfibers, including ultra-fine microfibers (with an average diameter of about 3 microns or less). A description of an exemplary process of making ultra-fine microfibers may be found in, for example, U.S. Pat. No. 5,213,881 to Timmons, et al. Meltblown fibers may be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

“Water impermeable,” when used in describing a layer or multi-layer laminate means that water or water vapor will not pass to any appreciable extent through the layer or laminate, under ordinary use conditions, in a direction generally perpendicular to the plane of the layer or laminate at the point of liquid contact.

“Water permeable” refers to any material that is not water impermeable.

These terms may be defined with additional language in the remaining portions of the specification.

Description

Various problems associated with skin or tissue may diminish the health and/or hygiene of a person or animal. For example, dryness of the skin of the hands, feet, extremities, joints, or other parts of a body is a common problem, especially when skin is exposed to cold and/or dry conditions. This may be especially true for older individuals. Various formulations designed to moisturize skin are often used to address this problem. Some formulations require a substantial amount of time to deliver the anticipated benefit. For many currently available formulations, any resulting skin benefit may last a relatively short period of time after the formulation has been applied. The present invention characterizes an appliance for delivering a formulation or composition to tissue or skin.

One example of such a composition is a skin-care formulation for moisturizing skin. Such formulations typically work through at least one of two mechanisms: occlusivity or humectancy. Skin-care formulations relying on occlusivity form a relatively water-vapor-impermeable film on and/or in a skin surface. This occlusive film results in the accumulation of water underneath the film as the skin undergoes the natural process of trans-epidermal water loss. One advantage of the occlusivity approach includes the ability to provide a skin-moisturization benefit for an extended period of time. Occlusive ingredients are typically hydrophobic in nature and are generally not easily washable, which contribute to their ability to provide long-term moisturization of skin. Formulations with ingredients having occlusive properties (such as peterolatum, waxes, vegetable oils, mineral oil, etc.) are perceived by some as having undesirable aesthetic and/or feel attributes. Occlusive formulations may also lack the ability to provide quick moisturization as such formulations depend on the relatively slow process of water accumulation due to trans-epidermal water loss to deliver the moisturizing benefit.

Humectant formulations have the ability to attract water vapor (moisture) from the atmosphere and bring it to the skin surface, which results in increased skin hydration and alleviation of dryness. This process of attracting moisture is frequently referred to as “hygroscopicity”. Humectant formulations have the advantage of delivering a moisturization benefit and dryness relief within a short period of time. Humectant formulations are typically hydrophilic (as noted above, “water loving”) in nature and generally contain a significant amount of water. Such formulations are generally perceived as having a light, pleasant feel (i.e., light in weight and/or viscosity) on the skin and typically are aesthetically preferred by the user (relative to skin-care compositions that function by occlusivity). Examples of humectant ingredients include glycerin, urea, sodium lactate, polysaccharides, and the like. Unlike occlusive formulations, humectant formulations generally lack the ability to provide moisturization over an extended period of time.

In one preferred aspect of the present invention, an aqueous formulation comprising a humectant is applied to the inner surface of an appliance such as a glove, sock, or sleeve. The appliance comprises a water-impermeable layer, such as a film, that prevents, reduces, or minimizes transmission of water through the appliance to the external environment. The water-impermeable layer is sandwiched between two fibrous layers (e.g., nonwoven materials). Without being bound to a particular theory, we believe an appliance comprising such a formulation realizes benefits of both humectancy and occlusivity without their respective disadvantages. The humectant formulation on the inside of the appliance provides the aesthetically pleasing feel to the appliance user and delivers the initial quick moisturization benefit to the skin. The occlusive nature of the water-impermeable film in the product contributes to a longer-term moisturization effect. Furthermore, the fibrous layers give the appliance a cloth-like feel and appearance. Also, undulations in the inner fibrous layer (which contacts skin or tissue of the user) helps contain the formulation or composition that is applied to the inner fibrous layer. As discussed elsewhere, these undulations (rugosity) may be effected, in whole or in part, by attaching the fibrous layers to the water-impermeable layer at discrete points or locations while the water-impermeable layer is in a stretched condition. When the resulting laminate is allowed to contract, the fibrous layers are gathered to enhance or produce undulations in the fibrous layers.

Representative Substrates for Constructing an Appliance of the Present Invention

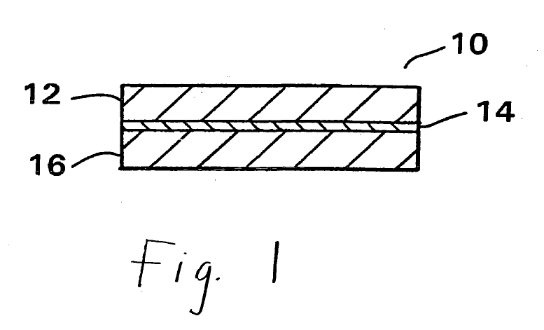

A substrate used to make an appliance of the present invention will generally have 3 layers: a water-impermeable layer, such as a film, sandwiched between two fibrous layers, such as nonwoven materials. An example of such a substrate 10 is depicted in FIG. 1, which representatively illustrates a water-impermeable layer 14 attached to an outer fibrous layer 12 and an inner fibrous layer 16. The material for the outer fibrous layer 12 may be any material that provides for a cloth-like appearance (as opposed to, for example, a smooth or rubbery appearance as in neoprene rubber glove). The material for the inner fibrous layer 16 may be any material that is fibrous in nature, such as a nonwoven material. The inner fibrous layer should possess an uneven, undulating surface to help contain the formulation or composition applied to the surface of the inner fibrous layer 16. As noted above, the rugosity of this inner material can be achieved or enhanced by attaching the inner fibrous layer 16 to the water-impermeable layer 14 at discrete points or locations (e.g., by thermally point bonding the materials together, as is discussed in more detail below) while the water-impermeable layer 14 is in a stretched condition. When the water-impermeable layer 14 (and, therefore, the resulting laminate) is allowed to relax, the inner fibrous layer 16 is gathered to produce undulations in the inner fibrous layer. Of course, both the inner fibrous layer 16 and the outer fibrous layer 12 are gathered in this way if they are attached to the water-impermeable layer 14 at discrete points or locations while the water-impermeable layer 14 is in a stretched condition (and then allowed to relax).

The inner and outer fibrous layers may be the same or may be different. Generally the water-impermeable layer 14 is elastomeric, with the resulting substrate 10 able to stretch and conform to a hand, foot, extremity, or other body region to which the appliance is applied.

If a nonwoven material is used to make the inner and outer fibrous layers, then commercially available thermoplastic polymeric materials can be advantageously employed in making the fibers or filaments from which the outer fibrous layer 12 and inner fibrous layer 16 are formed. As used herein, the term “polymer” shall include, but is not limited to, homopolymer, copolymers, such as, for example, block, graft, random and alternating copolymers, terpolymers, etc., and blends and modifications thereof. Moreover, unless otherwise specifically limited, the term “polymer” shall include all possible geometric configurations of the material, including, without limitation, isotactic, syndiotactic, random and atactic symmetries. As used herein, the terms “thermoplastic polymer” or “thermoplastic polymeric material” refer to a long-chain polymer that softens when exposed to heat and returns to the solid state when cooled to ambient temperature. Exemplary thermoplastic materials include, without limitation, polyvinyl chlorides, polyesters, polyamides, polyfluorocarbons, polyolefins, polyurethanes, polystyrenes, polyvinyl alcohols, caprolactams, and copolymers of the foregoing.

Nonwoven webs that can be employed as the nonwoven layers 12 and 16 of the present invention can be formed by a variety of known forming processes, including spunbonding, airlaying, meltblowing, or bonded carded web formation processes. Spunbond nonwoven webs are made from melt-spun filaments. As used herein, the term “meltspun filaments” refers to small diameter fibers and/or filaments which are formed by extruding a molten thermoplastic material as filaments from a plurality of fine, usually circular, capillaries of a spinneret with the diameter of the extruded filaments then being rapidly reduced, for example, by non-eductive or eductive fluid drawing or other well known spunbonding mechanisms. Lastly, the melt-spun filaments are deposited in a substantially random manner onto a moving carrier belt or the like to form a web of substantially continuous and randomly arranged, melt-spun filaments. Spunbond filaments generally are not tacky when they are deposited onto the collecting surface. The production of spunbond nonwoven webs is described in U.S. Pat. No. 4,340,563 to Appel et al., U. S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,538 to Peterson, and U.S. Pat. No. 3,542,615 to Dobo et al., all of which are incorporated herein by reference. The melt-spun filaments formed by the spunbond process are generally continuous and have average diameters larger than 7 microns based upon at least 5 measurements, and more particularly, between about 10 and 100 microns. Another frequently used expression of fiber or filament diameter is denier, which is defined as grams per 9000 meters of a fiber or filament.

Spunbond webs generally are stabilized or consolidated (pre-bonded) in some manner immediately as they are produced in order to give the web sufficient integrity and strength to withstand the rigors of further processing into a finished product. This pre-bonding step may be accomplished through the use of an adhesive applied to the filaments as a liquid or powder which may be heat activated, or more commonly, by compaction rolls. As used herein, the term “compaction rolls” means a set of rollers above and below the nonwoven web used to compact the web as a way of treating a just produced, melt-spun filament, particularly spunbond, web, in order to give the web sufficient integrity for further processing, but not the relatively strong bonding of later applied, secondary bonding processes, such as through-air bonding, thermal bonding, ultrasonic bonding and the like. Compaction rolls slightly squeeze the web in order to increase its self-adherence and thereby its integrity.

An exemplary secondary bonding process utilizes a patterned roller arrangement for thermally bonding the spunbond web. The roller arrangement typically includes a patterned bonding roll and a smooth anvil roll which together define a thermal patterning bonding nip. Alternatively, the anvil roll may also bear a bonding pattern on its outer surface. The pattern roll is heated to a suitable bonding temperature by conventional heating means and is rotated by conventional drive means, so that when the spunbond web passes through the nip, a series of thermal pattern bonds is formed. Nip pressure within the nip should be sufficient to achieve the desired degree of bonding of the web, given the line speed, bonding temperature and materials forming the web. Percent bond areas within the range of from about 10 percent to about 20 percent are typical for such spunbond webs.

The water-impermeable layer 14 can be formed of any film that can be suitably bonded or attached to top and bottom layers 12 and 16 respectively to yield a substrate 10 having the performance characteristics and features described herein. A suitable class of film materials includes a thermoplastic elastomeric polyolefin polymer. These (and other) components can be mixed together, heated and then extruded into a mono-layer or multi-layer film using any one of a variety of film-producing processes known to those of ordinary skill in the film processing art. Such film-making processes include, for example, cast embossed, chill and flat cast, and blown film processes. Typically the water-impermeable layer 14 will be attached to the outer fibrous layer 12 and inner fibrous layer 16 by thermally bonding the three layers together at discrete points (see, e.g., discussion in preceding paragraph as well as U.S. Pat. No. 6,037,281, entitled “Cloth-Like, Liquid-Impervious, Breathable Composite Barrier Fabric,” to Mathis, et al.). As noted above, the two fibrous layers may be bonded or attached to the water-impermeable layer at discrete locations while the water-impermeable layer is in a stretched condition, thereby producing undulations when the resulting laminate is in a relaxed condition. Other known means for bonding and laminating the water-impermeable layer 14 to fibrous layers 12, 16 may be used, provided the resulting substrate 10 has the required properties described herein. For example, the three layers may be adhesively bonded to one another.

Other additives and ingredients may be added to the film layer 14 provided they do not significantly interfere with the ability of the film layer to function in accordance with the teachings of the present invention. Such additives and ingredients can include, for example, antioxidants, stabilizers, and pigments. In addition to the polyolefin polymer, the film layer 14 can also include a filler. As used herein, a “filler” is meant to include particulates and other forms of materials which can be added to the film polymer extrusion blend and which will not chemically interfere with the extruded film but which are able to be uniformly dispersed throughout the film. Generally, the fillers will be in particulate form and may have a spherical or non-spherical shape with average particle sizes in the range of about 0.1 to about 7 microns. Both organic and inorganic fillers are contemplated to be within the scope of the present invention provided that they do not interfere with the film formation process, or the ability of the film layer to function in accordance with the teachings of the present invention. Examples of suitable fillers include calcium carbonate (CaCO₃), various kinds of clay, silica (SiO₂), alumina, barium carbonate, sodium carbonate, magnesium carbonate, talc, barium sulfate, magnesium sulfate, aluminum sulfate, titanium dioxide (TiO₂), zeolites, cellulose-type powders, kaolin, mica, carbon, calcium oxide, magnesium oxide, aluminum hydroxide, pulp powder, wood powder, cellulose derivatives, chitin and chitin derivatives. A suitable coating, such as, for example, stearic acid, may also be applied to the filler particles.

As mentioned herein, film layer 14 may be formed using any one of the conventional processes known to those familiar with film formation. The polyolefin polymer and any optional ingredients (e.g., filler) are mixed in and then heated and extruded into a film.

The film layer used in the example of the present invention described below is a mono-layer film, however, other types, such as multi-layer films, are also considered to be within the scope of the present invention provided the forming technique is compatible with films described herein.

The preceding paragraphs describe some examples of a substrate that may be used in an appliance of the present invention. For additional examples, see, e.g., U.S. Pat. No. 6,037,281, entitled “Cloth-Like, Liquid-Impervious, Breathable Composite Barrier Fabric,” to Mathis, et al.; U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference in a manner consistent herewith. Any 3-layer substrate may be used, so long as the outer fibrous layer presents a cloth-like appearance and feel; the inner fibrous layer is sufficiently rugose to help contain the formulation or composition applied to the interior of the appliance (to minimize leakage of the formulation or composition from the appliance); and the water-impermeable layer—such as a film—is capable of minimizing evaporation or transfer of water through the appliance.

Representative Appliance Configurations

One or more substrates, such as those described above, may be configured into the form of a glove, mitten, sock, sleeve, patch, or other article designed to be fitted to a part of the body. Generally the appliance will be made by cutting a substrate into appropriate pieces such that the pieces, when attached to one another, form an appliance having an interior volume into which a portion of a body may be inserted.

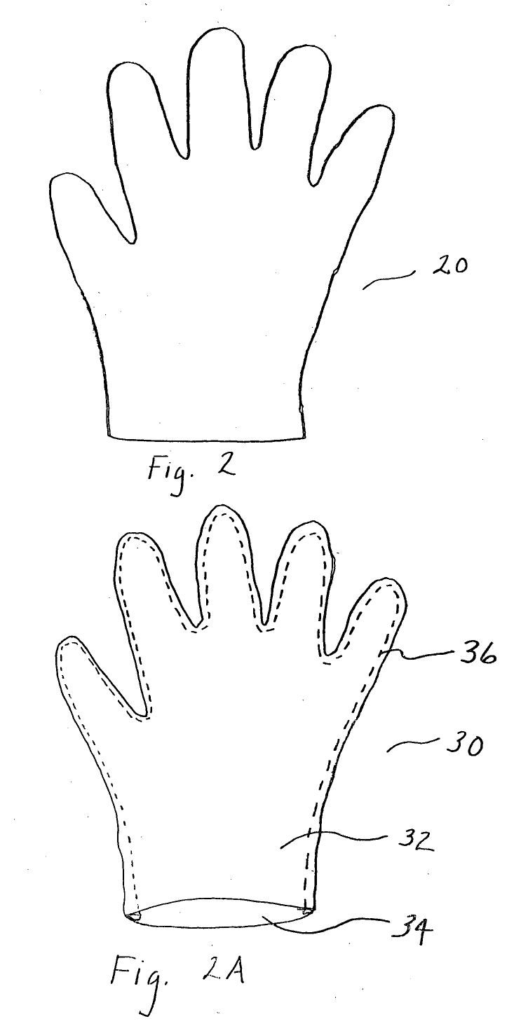

FIG. 2 representatively depicts a substrate 20 cut so that the piece (or substrate) defines a perimeter in the shape of a human hand. FIG. 2A representatively depicts an appliance 30 comprising a first piece (or substrate) 32 attached to a second piece (or substrate) 34 at a location proximate to the perimeters of these two substrates. In this representative illustration, the two substrates are attached to one another mechanically by sewing the pieces together at a location proximate to the perimeters of the two substrates. The resulting appliance was then inverted so that the seam 36 formed by sewing the substrates together is on the interior of the appliance. Of course the finished appliance need not be inverted; the seam can remain on the exterior of the appliance. Note, too, that the individual pieces need not be joined in a way that produces a seam. The edges of the individual pieces may be butted together, and then, for example, joined and/or welded together using a solvent. Alternatively, the individual pieces may be butted together, and another material, such as an adhesive or an adhesive tape, used to join the pieces together.

Individual pieces (or substrates) may be cut into a variety of shapes and sizes. Rather than the glove depicted in FIGS. 2 and 2A, the pieces may be cut so that the resulting appliance is in the shape of a tube, sleeve, mitten, sock, or the like. Any shape is possible, so long as the resulting appliance defines an interior volume into which a user may insert a portion of his or her body (e.g., a finger, toe, hand, foot, wrist, forearm, etc.) such that a composition applied to the interior surface of the appliance may be transferred to skin or tissue in contact with the interior surface of the appliance.

The individual substrates or pieces need not be sewn together. The individual pieces or substrates may also be joined ultrasonically, thermally, adhesively, cohesively, using tape, by fusing the materials together (e.g., by using an appropriate solvent), by welding the materials together, or by other approaches. So long as the individual pieces or substrates remain attached or connected during normal use of the appliance, and attachment or connection is such that the composition or formulation on the interior surface of the appliance is contained within the appliance (i.e., there is minimal or no leakage of the formulation or composition), any connection or attachment may be used.

Alternatively, a substrate could be prepared in the form of a rectangle, oval or other shape. An adhesive capable of adhering to skin could then be applied to all or part of the perimeter of the shape such that the appliance could be releasably adhered to skin. The composition to be transferred to skin could then be coated or deposited on the surface of the appliance that will contact skin or tissue.

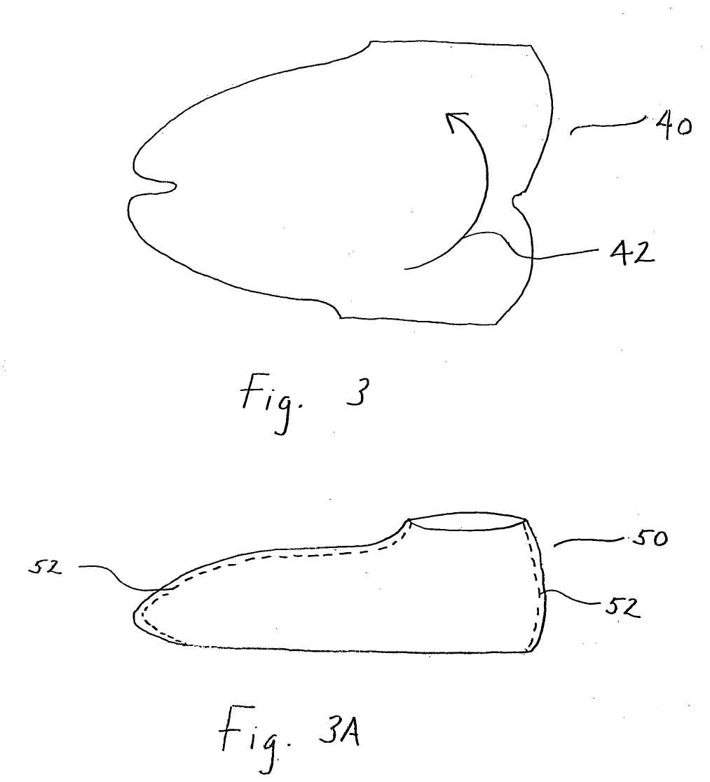

Note, too, that an appliance may be formed from a single piece of substrate. FIG. 3 representatively illustrates a substrate 40 that has been cut in a way that a foot-shaped appliance may be formed by folding the substrate back on itself (as shown by arrow 42; the bottom half of the shape is folded upward, and on top of, the top half of the shape). FIG. 3A representatively illustrates such a foot-shape appliance 50 and the resulting seams 52 formed when the substrate 40 (from FIG. 3) is folded back, and attached to, itself. In this representative embodiment, the foot-shape appliance was inverted after the substrate was attached to itself so that the seams were on the inside of the appliance. As with two (or more) pieces that may be joined together to form an appliance of the present invention, a single piece may be joined to itself using any of the approaches discussed above.

Representative Formulations or Compositions for Use With an Appliance of the Present Invention

Formulations or compositions that may be used with an appliance of the present invention include emulsifiers, surfactants, viscosity modifiers, natural moisturizing factors, antimicrobial actives, pH modifiers, enzyme inhibitors/inactivators, suspending agents, pigments, dyes, colorants, buffers, perfumes, antibacterial actives, antifungal actives, pharmaceutical actives, film formers, deodorants, opacifiers, astringents, solvents, organic acids, preservatives, drugs, vitamins, aloe vera, and the like.

In some versions of the invention, a clinically beneficial additive of the formulation or composition may either interact directly with epithelial tissue at the cellular level to provide a benefit to the skin, or alternatively, may interact with components at or near the skin surface in order to provide a benefit to the skin.

In one embodiment, the clinically beneficial additive may be an emollient, which is herein defined as an agent that helps restore dry skin to a more normal moisture balance. Emollients act on the skin by supplying fats and oils that blend in with skin, making it pliable, repairing some of the cracks and fissures in the stratum corneum, and forming a protective film that traps water in the skin. Emollients that may be suitable for use with the present invention include beeswax, butyl stearate, cermides, cetyl palmitate, eucerit, isohexadecane, isopropyl palmitate, isopropyl myristate, mink oil, mineral oil, nut oil, oleyl alcohol, petroleum jelly or petrolatum, glyceral stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, certain hydrogel emollients, various lipids, decyl oleate and castor oil.

A preferred clinically beneficial additive is a humectant, which is herein defined to be an agent that supplies the skin with water by attracting moisture from the air and holding it on the skin. Humectants that may be suitable for use with the present invention include alanine, glycerin, PEG, propylene glycol, butylenes glycol, glycerin (glycol), hyaluronic acid, Natural Moisturizing Factor (a mixture of amino acids and salts that are among the skin's natural humectants), saccharide isomerate, sodium lactate, sorbitol, urea, and sodium PCA.

Other clinically beneficial agents that may be suitable for use with the present invention include antioxidants, a unique group of substances that protect a body or other objects from oxidizing. Antioxidants prevent or slow the oxidation process, thereby protecting the skin from premature aging. Exemplary antioxidants for use in the present invention include ascorbic acid ester, vitamin C (ascorbic acid), vitamin E (lecithin), Alpha-Glycosyl Rutin (AGR, or Alpha Flavon, a plant-derived antioxidant), and coenzyme Q10 (also known as ubiquinone).

Other clinically beneficial agents which may be delivered to the skin during use include chelating agents, such as EDTA; absorptive/neutralizing agents, such as kaolin, hectorite, smectite, or bentonite; other vitamins and vitamin sources and derivatives, such as panthenol, retinyl palmitate, tocopherol, and tocopherol acetate; and anti-irritants such as chitin and chitosan.

Additional examples of beneficial agents include skin conditioners, which are herein defined as agents that may help the skin retain moisture, improve softness, or improve texture. Skin conditioners include, for example, amino acids, including alanine, serine, and glycine; allantoin, keratin, and methyl glucose dioleate; alpha-hydroxy acids, including lactic acid and glycolic acid, which act by loosening dead skin cells from the skin's surface; moisturizers (agents that add or hold water in dry skin), including echinacea (an extract of the coneflower plant), shea butter, and certain silicones, including cyclomethicon, dimethicone, and simethicone.

Other examples of beneficial botanical agents, extracts, or other materials that may be suitable for use with the present invention include almonds, chamomile extracts such as bisabolol (believed to relieve irritation, swelling and itching in the skin), elder flowers, honey, safflower oil, and elastin (safflower oil and elastin are believed to aid in retaining skin elasticity).

In addition to one or more clinically beneficial additives, other additives may be included in the formulation or composition. For example, a silicone polymer may be included to improve the slip characteristics of the elastomeric article. Possible silicone polymers include reactive silicones, non-reactive silicones, or a mixture of reactive and non-reactive silicones. Suitable silicones may include, for example, aminosilicones, polyether-modified amino silicones, amino-substituted siloxanes having terminal hydroxy groups, epoxy silicones, quaternary silicones, dimethicone, silicone polyethers, polyether epoxy silicones, silanol fluids, polysiloxy linoleyl pyrrolidone phospholipids, and combinations of possible silicones.

Other additives may be included, for example, glucose derived polymers, or mixtures containing glucose derived polymers (e.g., lauryl glucoside available from Cospha under the trade designation Planteran PS 400), silica, silica dispersions, wetting agents, and preservatives (i.e., parabens, such as methylparaben and propylparaben). In one embodiment, the personal-care composition may include emulsion stabilizers. Exemplary emulsion stabilizers include aluminum stearate, magnesium sulfate, hydrated silica, and ozokerite.

In another embodiment a beneficial agent may be held in the formulation or composition in liposomes. A liposome is a vehicle for delivering agents to the skin. More specifically, a liposome is a microscopic sphere formed from a fatty compound, a lipid, surrounding a water-based agent, such as a moisturizer or an emollient. When the liposome is rubbed into the skin, it releases the agent throughout the stratum corneum.

In another embodiment, the beneficial agent may be present in the carrier in the form of a microencapsulant. A microencapsulant is a sphere of an emollient surrounded by a gelatin membrane that prevents the emollient from reacting with other ingredients in the coating composition and helps distribute the emollient more evenly when pressure is applied and the membrane is broken. The process of forming these beads is called microencapsulation and is generally known in the art.

The formulation or composition of the present invention may be applied to the appliance as an aqueous solution, a dispersion, or an emulsion. In one embodiment, an aqueous composition may be formed including from about 4.5% to about 6% by weight of a humectant. In other embodiments, the humectant may be present at 30% or more by weight. In some other embodiments, the humectant may be present at about 10 to about 20% by weight. In still other embodiments, the humectant is present at about 5 to about 40% by weight. This composition may then be applied to the interior surface of an appliance of the present invention.

In one embodiment, the personal-care composition may be applied as an emulsion. In one embodiment, the formulation or composition may be applied to the surface of the appliance as a micro-emulsion. A micro-emulsion is a particularly fine-particle emulsion that can be applied in a spray form. The particle size of a micro-emulsion is generally less than about one micron, whereas traditional emulsions demonstrate particle sizes of greater than about 50 microns.

The components of a formulation or composition may be applied in combination or separately to the surface of the appliance. For example, a 100% humectant composition may be applied, followed by another 100% beneficial additive composition, such that the two (or more) separate applications together form the coating of the appliance. In such a manner, layers of additives may be built up on the surface of the appliance.

The coating may be deposited on the interior surface of the appliances by any suitable method. For example, the appliances may be dipped in the coating. In an alternative embodiment, the appliances may be tumbled in the coating. In various embodiments, the coating may be applied to the surface of the appliance through dipping, immersion, spraying, patting, printing, or any other application method known in the art.

In one embodiment, the coating may be sprayed onto a skin-contacting surface of the appliance. For instance, appliances may be placed in a tumbling apparatus while a solution of the coating is sprayed on the gloves. In one embodiment, the spraying process may be repeated. For instance, the spraying process may be repeated up to about twenty times to coat the inner surface of the gloves. In one embodiment, the spraying process may be carried out for a total of between about ten and about twenty times.

Representative Marketing and/or Packaging of Appliances of Present Invention

The manufacturer of an appliance of the present invention may fashion messages, statements, or copy to be transmitted to a purchaser, consumer, or user of said appliance. Such messages, statements, or copy may be fashioned to help facilitate or establish an association in the mind of a user of the appliance between an appliance of the present invention, or use thereof, and one or more mental states, psychological states, or states of well being. The communication, statements, or copy may include various alphanumeric strings, including, for example: relax, peace, energy, energize, sex, sensuality, sensual, spirit, spiritual, clean, fresh, mountain, country, zest, sea, sky, health, hygiene, water, waterfall, moisture, moisturize, or derivatives or combinations thereof. These alphanumeric strings may be used either alone, adjacent to, or in combination with, other alphanumeric strings. The communication, statements, message, or copy could take the form of (i.e., be embodied in a medium such as) a newspaper advertisement, a television advertisement, a radio or other audio advertisement, items mailed directly to addressees, items emailed to addresses, Internet Web pages or other such postings, free standing inserts, coupons, various promotions (e.g., trade promotions), co-promotions with other companies, copy and the like, boxes and packages containing the product (in this case an appliance of the present invention), and other such forms of disseminating information to consumers or potential consumers. Other exemplary versions of such communications, statements, messages, and/or copy may be found in, for example, U.S. Pat. Nos. 6,612,846 and 6,896,521, both entitled “Method for Displaying Toilet Training Materials and Display Kiosk Using Same”; co-pending U.S. application Ser. No. 10/831476, entitled “Method of Enunciating a Pre-Recorded Message Related to Toilet Training in Response to a Contact”; co-pending U.S. application Ser. No. 10/956763, entitled “Method of Manufacturing and Method of Marketing Gender-Specific Absorbent Articles Having Liquid-Handling Properties Tailored to Each Gender”; each of which is incorporated by reference in their entirety in a manner consistent herewith.

It should be noted that when associating statements, copy, messages, or other communications with a package (e.g., by printing text, images, symbols, graphics, color(s), or the like on the package; or by placing printed instructions in the package; or by associating or attaching such instructions, a coupon, or other materials to the package; or the like) containing appliances of the present invention, the materials of construction of said package may be selected to reduce, impede, or eliminate the passage of water or water vapor through at least a portion of the package. As noted above, a preferred composition for an appliance of the present invention comprises water and a humectant. Therefore packages, containers, envelopes, bags, and the like that reduce, minimize, or eliminate the evaporation or transmission of water or water vapor from appliances contained therein is beneficial. Furthermore, appliances may be individually wrapped in containers, packets, envelopes, bags, or the like that inhibit, reduce, or eliminate the passage or transmission of water or water vapor from appliances contained therein. For purposes of this application, “packages,” “containers,” “envelopes,” “bags,” “packets,” and the like are interchangeable in the sense that they refer to any material adapted to enclose and hold either individual appliances (as in, for example, an individual packet containing a single appliance), or a plurality of appliances (as in a flexible bag made of film containing a plurality of appliances, whether or not each of the individual appliances are enclosed and held in a separate material-such as individual packets).

These and other modifications and variations to the present invention may be practiced by those of ordinary skill in the art, without departing from the spirit and scope of the present invention, which is more particularly set forth in the appended claims. Furthermore, those of ordinary skill in the art will appreciate that the foregoing description is by way of example only, and is not intended to limit the invention so further described in such appended claims.

### EXAMPLES

### Example 1

### Representative Example of a Personal-Care Composition

An exemplary personal-care composition was prepared having the ingredients/components and proportions identified below:

| Component | Weight % | Supplier | Address |
|---|---|---|---|
| Water | 66.1 | N/A | N/A |
| Emulgade CM | 20.0 | Cognis | 300 Brookside Ave, |
| | | | Ambler, PA 19002 |
| Glycerin | 4.0 | Glenn Corp. | 4886 Highway 61 N, |
| (99.7% USP) | | | |
| | | | St. Paul, MN 55110 |
| Hispagel 200 | 4.8 | Cognis | 300 Brookside Ave, |
| | | | Ambler, PA 19002 |
| Sepigel 501 | 3.2 | Seppic | 30, Two Bridges |
| | | | Road, Fairfield, |
| | | | New Jersey 07004 |
| Mackernium-007 | 1.0 | McIntyre Group | 24601 Governors |
| | | | Highway, University |
| | | | Park, IL 60466 |
| Paragon III | 0.5 | McIntyre Group | 24601 Governors |
| | | | Highway, University |
| | | | Park, IL 60466 |
| Fragrance | 0.05 | | |
| Tween 40 | 0.05 | Uniquema | 76 East 24^{th} St, |
| | | | Paterson, NJ 07544 |
| Sodium | 0.3 | Sigma-Aldrich | 3050 Spruce Street, |
| citrate 20% | | | St. Louis, MO 63103 |

The recited proportions of water, glycerin, Emulgade CM, Mackernium-007, and Paragon III were mixed together a Lightnin Labmaster mixer LIU10F (135 Mt. Read Blvd., Rochester, N.Y.). Tween 40 and the fragrance were mixed separately in a small container using a spatula. The fragrance/Tween 40 mixture was then mixed into the mixture containing water, glycerin, and the other ingredients identified above. Hispagel 200 and Sepigel 501 were then added in sequence to the resulting combination in an Ultra-Turrax T50 Basic high-shear homogenizer (IKA® Works, 2635 Northchase Pkwy. SE, Wilmington, N.C. 28405). Finally, pH of the formulation was adjusted by adding sodium citrate until a pH of 6.0 was achieved, as measured using a SevenMulti pH meter (Mettler-Toledo, 1900 Polaris Parkway, Columbus, Ohio, 43240).

### Example 2

### Representative Example of a Personal-Care Composition

An exemplary personal-care composition having the ingredients and proportions identified below was prepared:

| Component | Weight % | Supplier | Address |
|---|---|---|---|
| Water | 67.1 | N/A | N/A |
| Emulgade CM | 20.0 | Cognis | 300 Brookside Ave, |
| | | | Ambler, PA 19002 |
| Glycerin (99.7% | 4.0 | Glenn Corp. | 4886 Highway 61 N, |
| USP) | | | St. Paul, MN 55110 |
| Pentavitin | 4.0 | Pentapharm/ | 20 Glover Ave, |
| (saccharide | | CenterChem | Norwalk, CT 06850 |
| isomerate) | | | |
| Sepigel 501 | 2.5 | Seppic | 30, Two Bridges |
| | | | Road, Fairfield, New |
| | | | Jersey 07004 |
| Panthenol | 1.0 | Sigma-Aldrich | 3050 Spruce Street, |
| | | | St. Louis, MO 63103 |
| Paragon III | 0.5 | McIntyre Group | 24601 Governors |
| | | | Highway, University |
| | | | Park, IL 60466 |
| Keltrol CG | 0.3 | CPKelco | 1000 Parkwood |
| (Xanthan Gum) | | | Circle, Atlanta, GA |
| | | | 30339 |
| Fragrance | 0.2 | | |
| Tween 40 | 0.2 | Uniquema | 76 East 24^{th} St, |
| | | | Paterson, NJ 07544 |
| Sodium citrate | 0.2 | Sigma-Aldrich | 3050 Spruce Street, |
| 20% | | | St. Louis, MO 63103 |

The recited proportion of xanthan gum was dispersed in water by thoroughly mixing the material in a Lightnin Labmaster mixer LIU10F (135 Mt. Read Blvd., Rochester, N.Y.) at a setting of 400 rpm until the gum was fully hydrated (approximately an hour). Glycerin, Emulgade CM, and Pentavitin, followed by panthenol and Paragon III were then mixed into the xanthan-gum/water mixture. Sepigel 501 was then added to the combination and homogenized using a high shear mixer (Ultra-Turrax T50 Basic, IKA® Works, 2635 Northchase Pkwy. SE, Wilmington, N.C. 28405). Tween 40 and the fragrance were mixed separately in a small container using a spatula. The fragrance/Tween 40 mixture was then mixed into the combination. Finally, pH of the formulation was adjusted by adding sodium citrate until a pH of 6.0 was achieved, as measured using a SevenMulti pH meter (Mettler-Toledo, 1900 Polaris Parkway, Columbus, Ohio, 43240).

### Example 3

### Representative Example of a Personal-Care Composition

An exemplary personal-care composition was prepared having the ingredients and proportions identified below was prepared:

| Component | Weight % | Supplier | Address |
|---|---|---|---|
| Water | 73.0 | N/A | N/A |
| Glycerin (99.7% | 5.0 | Glenn Corp. | 4886 Highway 61 N, |
| USP) | | | St. Paul, MN 55110 |
| Cognis IPP | 5.0 | Cognis | 300 Brookside Ave, |
| | | | Ambler, PA 19002 |
| Arlacel 165 | 3.0 | Uniquema | 76 East 24^{th} St |
| | | | Paterson, NJ 07544 |
| Petrolatum (Super | 3.0 | Crompton | 771 Old Saw Mill |
| White Protopet) | | | River Road, |
| | | | Tarrytown, NY 10531 |
| Lipex 512 (Shea | 2.0 | Jarchem | 414 Wilson Ave, |
| Butter) | | Industries | Newark, NJ 07105 |
| BioVera Oil | 2.0 | BioChemical | 498 Kingston Road, |
| (Aloe Vera) | | International | Satellite Beach, FL |
| | | | 32937 |
| DC 200 Fluid, | 2.0 | Dow Corning | PO Box 994, 2200 |
| 100 cst | | | West Salzburg Road, |
| | | | Midland, MI 48686 |
| Corpure Avocado | 1.0 | Croda | 7 Century Drive, |
| (Avocado Oil) | | | Parsippany, NJ |
| | | | 07054 |
| Cetyl Alcohol | 1.0 | Glenn Corp. | 4886 Highway 61 N, |
| (NF) | | | St. Paul, MN 55110 |
| Emerest 2400 | 0.5 | Cognis | 300 Brookside Ave, |
| (Glyceryl | | | Ambler, PA 19002 |
| Stearate) | | | |
| dl-alpha Tocopherol | 0.5 | Ruger | 1515 W. Blancke |
| Acetate (Vitamin E | | Chemical | Street, Linden, NJ |
| acetate) | | | 07036 |
| Paragon III | 0.5 | McIntyre | 24601 Governors |
| | | Group | Highway, University |
| | | | Park, IL 60466 |
| Actiphyte of | 0.5 | Active | 1097 Yates Street, |
| Chamomile AQ | | Organics | Lewisville, TX 75057 |
| (Chamomile Extract) | | | |
| Ketrol CG (Xanthan | 0.2 | CPKelco | 1000 Parkwood |
| Gum) | | | Circle, Atlanta, GA |
| | | | 30339 |
| Fragrance | 0.2 | | |
| Tween 40 | 0.2 | Uniquema | 76 East 24^{th} St, |
| | | | Paterson, NJ 07544 |
| Sodium Citrate 20% | 0.2 | Sigma-Aldrich | 3050 Spruce Street, |
| solution | | | St. Louis, MO 63103 |
| Versene NA | 0.1 | Dow Chemical | PO Box 1206, |
| (Disodium EDTA) | | | Midland, MI 48642 |
| BHT | 0.1 | Universal | 33 Truman Drive |
| | | Preserv-A- | South, Edison, NJ |
| | | Chem | 08817 |

The recited proportion of xanthan gum was dispersed in water by thoroughly mixing the material in a Lightnin Labmaster mixer LIU10F (135 Mt. Read Blvd., Rochester, N.Y.) at a setting of 400 rpm until the gum was fully hydrated for approximately one hour. Each of the aqueous ingredients—glycerin, Paragon III, Actiphyte of Chamomile AQ, and Versene NA—were then added to the water phase. (This complete formulation is an oil-in-water (o/w) emulsion where all water-soluble ingredients are mixed separately and called “the water phase”; and the same is done for the oil-soluble ingredients denoted as the “oil phase.”) Tween 40 and the fragrance were mixed separately in a small container using a spatula. The fragrance/Tween 40 mixture was then mixed into the combination. The water phase was then heated to 76 degrees Celsius using a VWR hotplate (1310 Goshen Parkway, West Chester, Pa. 19380). Ingredients for the oil phase of the formulation were then mixed in a separate container using the Lightnin Labmaster and also heated to 76 degrees Celsius on a hotplate. The ingredients were Cognis IPP, Arlacel 165, Petrolatum, Lipex 512, BioVera Oil, DC 200 Fluid, Corpure Avocado, Cetyl Alcohol, Emerest 2400, dl-alpha tocopherol acetate, and BHT. The oil-phase mixture was then added to the water-phase mixture, both at the recited temperature of 76 degrees Celsius. The combination was mixed in a container at a setting of 400 rpm. After mixing at this speed for approximately 10 minutes, the rotational speed was increased to 470 rpm for an additional 5 minutes. Then the combination was homogenized for three minutes using a high-shear mixer (Ultra-Turrax T50 Basic, IKA® Works, 2635 Northchase Pkwy. SE, Wilmington, N.C. 28405). The combination was then allowed to cool down with the Lightnin Labmaster mixer LIU10F (135 Mt. Read Blvd., Rochester, N.Y.) set at a rotational speed of 400 rpm. Finally, pH of the formulation was adjusted by adding sodium citrate until a pH of 6.0 was achieved, as measured using a SevenMulti pH meter (Mettler-Toledo, 1900 Polaris Parkway, Columbus, Ohio, 43240).

### Example 4

### Representative Example of a Personal-Care Composition

An exemplary personal-care composition was prepared having the ingredients and proportions identified below was prepared:

| Component | % | Supplier | Address |
|---|---|---|---|
| Water | 63.7 | N/A | N/A |
| Emulgade CM | 20.0 | Cognis | 300 Brookside Ave, |
| | | | Ambler, PA 19002 |
| Glycerin | 4.0 | Glenn Corp. | 4886 Highway 61 N, |
| (99.7% USP) | | | St. Paul, MN 55110 |
| Hispagel 200 | 4.8 | Cognis | 300 Brookside Ave, |
| | | | Ambler, PA 19002 |
| Sepigel 501 | 3.2 | Seppic | 30, Two Bridges |
| | | | Road, Fairfield, New |
| | | | Jersey 07004 |
| Mackernium-007 | 1.0 | McIntyre Group | 24601 Governors |
| | | | Highway, University |
| | | | Park, IL 60466 |
| Paragon III | 0.5 | McIntyre Group | 24601 Governors |
| | | | Highway, University |
| | | | Park, IL 60466 |
| Panthenol | 0.5 | Sigma-Aldrich | 3050 Spruce Street, |
| | | | St. Louis, MO 63103 |
| Tindoerm A | 0.5 | Ciba Specialty | 4090 Premier Drive, |
| | | Chemicals | High Point, NC |
| | | | 27261 |
| Actiphyte of Aloe | 0.5 | Active Organics | 1097 Yates Street, |
| Vera extract 10 fold | | | Lewisville, TX 75057 |
| Actiphyte of | 0.25 | Active Organics | 1097 Yates Street, |
| Avocade | | | Lewisville, TX 75057 |
| Actiphyte of Jojoba | 0.25 | Active Organics | 1097 Yates Street, |
| Meal | | | Lewisville, TX 75057 |
| Fragrance | 0.2 | | |
| Tween 40 | 0.2 | Uniquema | 76 East 24^{th} St, |
| | | | Paterson, NJ 07544 |
| Tinoderm E | 0.1 | Ciba Specialty | 4090 Premier Drive, |
| | | Chemicals | High Point, NC |
| | | | 27261 |
| Sodium citrate 20% | 0.3 | Sigma-Aldrich | 3050 Spruce Street, |
| | | | St. Louis, MO 63103 |

The recited proportions of water, glycerin, Emulgade CM, Mackernium-007, Tinoderm A, Tinoderm E, Aloe Vera, Avocado, Jojoba Meal and Paragon III were mixed together a Lightnin Labmaster mixer LIU10F (135 Mt. Read Blvd., Rochester, N.Y.). Tween 40 and the fragrance were mixed separately in a small container using a spatula. The fragrance/Tween 40 mixture was then added to the previous combination. Hispagel 200 and Sepigel 501 were then added in sequence to the resulting combination in an Ultra-Turrax T50 Basic high-shear homogenizer (IKA® Works, 2635 Northchase Pkwy. SE, Wilmington, N.C. 28405). Finally, pH of the formulation was adjusted by adding sodium citrate until a pH of 6.0 was achieved, as measured using a SevenMulti pH meter (Mettler-Toledo, 1900 Polaris Parkway, Columbus, Ohio, 43240).

### Example 5

### Representative Appliances

Exemplary appliances were prepared in the following manner.

First, a water-impermeable layer was prepared by producing a cast film prepared from Kraton 6638 polymer resin (Kraton 6638 is a blend of 80% by weight Kraton 1730 styrene-(ethylene-propylene)-styrene-(ethylene-propylene) tetrablock copolymer from Kraton Polymers LLC, 7% by weight PETROTHANE NA601 polyethylene wax from Quantum Chemical Co., and 13% by weight REGALREZ 1126 tackifier from Eastman Chemical Co). As the film was being produced (at a 75 grams per square meter basis weight prior to stretching), it was stretched by about 400% along the dimension parallel to the direction of travel of the film. To each side of the film, necked polypropylene spunbond materials, each having a basis weight of 0.8 ounces per square yard, were attached. This was accomplished by first spraying a hot-melt adhesive, #2840 (available from Ato-Findley), at an add-on level of 2 grams per square meter, and heated to a temperature of 365 degrees Fahrenheit, to each side of the spunbond materials that were to contact the film. All three materials were directed to a nip between to rolls such that the spunbond materials were adhesively bonded to the film, thereby creating a 3-layer laminate. The laminate was then allowed to retract, thereby gathering the spunbond materials.

Once the substrate was prepared, it was cut into individual pieces in the shape of hand and foot (similar to the shapes representatively depicted in FIGS. 2, 2A, 3, and 3A above). The individual pieces were then sewn together to make either a glove into which a user could insert his or her hand, or a sock into which a user could insert his or her foot.

A personal-care composition was then applied. Each of the formulations identified in Examples 1, 2, and 3 above were added to the socks and gloves. The formulations were applied to the exposed surfaces of the appliances (with the seam visible) using a syringe (4 grams) and a spatula was used to spread the formulation to cover all parts of the product. The product was then inverted back with the seams inside and placed in air-tight bags to prevent evaporation until the time of use.

### Example 6

### Evaluation of Representative Appliances

Exemplary appliances were evaluated to determine their effect on skin moisture. Forty participants (all females, age 18 to 60 years) were divided into two groups. All participants received both glove and sock appliances. Each glove appliance contained one of the three different formulations identified in Examples 1-3 above, or were untreated (control). Each sock appliance also contained one of the three different formulations identified in Examples 1-3 above, but did not include an untreated control (in the Table below, Glove A comprises the formulation described in Example 1; Glove B comprises the formulation described in Example 2; Glove C comprises the formulation described in Example 3; Sock A comprises the formulation described in Example 1; Sock B comprises the formulation described in Example 2; Sock C comprises the formulation described in Example 3). For each hand, one site (2×2 cm) was demarcated approximately 3 cm below the middle knuckle. For each foot, one site (2×2 cm) was demarcated on the inside heel, approximately 2 cm below the instep (arch of the foot) near the bottom of the foot. Cell 1 received a foot buff after wearing the sock product overnight. The foot buff contained 7 grams of cleansing formulation. The formulation was applied uniformly on the foot buff using a syringe. Water was used to activate the cleansing formulation and a trained technician conducted the buffing for a period of one minute. The feet were rinsed with water for 15 seconds prior to and following the buffing process. Cell 2 received a foot buff at the beginning of the study, following baseline assessments. Measurements were taken at eight different time points (see Table below: after 15 minutes of wear, after 2 hours of wear, 2 hours after the appliance was removed, 4 hours after the appliance was removed, after 10 hours (overnight) of wear, 2 hour post wear day 2, and 4 hour post wear on day 2; “D1” denotes day 1, and “D2” denotes day 2).Subjects were required to acclimate to the environmental conditions of the room for at least 15 minutes prior to each measurement, exposing the bottom of their feet and their hands to the air. During wear, the subjects were provided standard slide sandals (“flip-flops”) to wear when walking in place for their normal shoes. Otherwise, subjects were instructed not to place any socks or other coverings on their feet. Nothing was worn over the test gloves. After 2 hours of wear, both the gloves and socks were removed (post-wear) and measurements were taken 2 hours and 4 hours post-wear (after product removal). Fresh pairs of gloves and socks were provided to use during the overnight (10 hour) wear period.

Skin capacitance was used to measure skin moisture levels. The measurements were taken at each demarcated site (hands and feet) using a NOVA DPM 9003® (NOVA Technologies, Gloucester, Mass., USA). The instrument's probe was placed at the tests sites and measurements taken, in triplicate. The values presented in the Table below are dimensionless and correlate positively with the amount of moisture in skin (i.e., a larger value indicates a higher skin moisture content). The values equate to the change from a baseline value taken with the instrument prior to a subject applying and wearing a glove or sock. Thus a positive value means a higher moisture content than the baseline value; a negative value means a lower moisture content than the base line value. The mean for each set of measurements was recorded on a data recording form and used in calculations. The study was conducted in an environmentally controlled room (70°+3° Fahrenheit, 40%+10% Relative Humidity). All formulations resulted in a statistically significant increase in the skin moisture levels. The increase in moisturization level was significant, in most cases, after only 15 minutes of product wear. Generally, the moisturization level was still significantly higher than baseline 2 hours and after 4 hours after removal of the product.

NOVA DPM: Change from Baseline

| | | | Time Point (Estimated Change from Baseline) | | | | | | |
|---|---|---|---|---|---|---|---|---|---|
| | | | | | | 4 Hr. | | | |
| | | | | | | Post | | 2 Hr. | |
| | | | 15 Min. | 2 Hr. | 2 Hr. Post | Wear | 10 Hour | Post | 4 Hr. Post |
| Type | Cell | Example | Wear (D1) | Wear (D1) | Wear (D1) | (D1) | Wear (D2) | Wear (D2) | Wear (D2) |
| Glove | 1 | Glove A | 28.9 | 100.9 | 88.7 | 109.0 | 251.5 | 103.7 | 49.3 |
| | | Glove B | 58.1 | 84.5 | 101.3 | 101.4 | 195.9 | 114.5 | 64.3 |
| | | Glove C | 94.5 | 152.2 | 134.8 | 111.7 | 251.1 | 129.2 | 78.3 |
| | | Untreated | 16.7 | 41.8 | 60.8 | 61.2 | 37.9 | 30.1 | 28.4 |
| | 2 | Glove A | 45.1 | 163.1 | 94.5 | 45.0 | 180.9 | 105.1 | 106.0 |
| | | Glove B | 51.6 | 186.0 | 76.6 | 56.3 | 186.8 | 114.6 | 135.3 |
| | | Glove C | 101.3 | 193.0 | 89.7 | 67.9 | 226.1 | 144.5 | 98.3 |
| | | Untreated | 7.7 | 28.0 | 3.3 | −7.0 | 2.0 | 7.6 | 11.1 |
| Sock | 1 | Sock A | 15.6 | 53.0 | 82.8 | 98.2 | 130.9 | 15.3 | 25.6 |
| | | Sock B | −29.3 | 38.6 | 84.8 | 137.6 | 194.2 | 30.1 | 52.8 |
| | | Sock C | 10.9 | 41.8 | 58.1 | 119.5 | 178.2 | −36.2 | 0.2 |
| | 2 | Sock A | 34.4 | 110.1 | 112.8 | 86.6 | 93.4 | 70.4 | 80.2 |
| | | Sock B | 6.6 | 81.5 | 87.2 | 89.3 | 77.5 | 57.1 | 92.2 |
| | | Sock C | 40.2 | 126.0 | 130.7 | 87.3 | 135.2 | 66.7 | 95.3 |

We claim:

1. An appliance adapted to transfer a composition from the interior surface of the appliance to the skin of a wearer of the appliance, the appliance comprising:
a first substrate having a perimeter, the first substrate comprising a first water-impermeable layer sandwiched between, and attached to, a first inner fibrous layer and a first outer fibrous layer;
a second substrate having a perimeter, the second substrate comprising a second water-impermeable layer sandwiched between, and attached to, a second inner fibrous layer and a second outer fibrous layer, wherein the first substrate and the second substrate are attached to one another in a way that defines an interior volume into which a wearer of the appliance may insert a portion of his or her body;
a composition deposited on at least a portion of the first inner fibrous layer, the second inner fibrous layer, or both, wherein at least a portion of the composition is adapted to be transferred from one or both inner fibrous layers to the skin of the wearer of the appliance during use.

2. The appliance of claim 1 wherein the perimeter of the first substrate, the perimeter of the second substrate, or the perimeters of the first and second substrates define a shape of a human hand.

3. The appliance of claim 1 wherein the composition is adapted to moisturize the skin of a user of the appliance.

4. The appliance of claims 3 wherein the composition comprises water and a humectant.

5. The appliance of claim 1 wherein the first inner fibrous layer and the second inner fibrous layer are nonwovens.

6. The appliance of claim 1 wherein the first inner fibrous layer, the first outer fibrous layer, or both are attached to the first water-impermeable layer at discrete locations.

7. The appliance of claim 6 wherein the second inner fibrous layer, the second outer fibrous layer, or both are attached to the second water-impermeable layer at discrete locations.

8. The appliance of claim 7 wherein the first inner fibrous layer, the second inner fibrous layer, the first outer fibrous layer, the second outer fibrous layer, or some combination thereof are gathered.

9. A package, the package comprising:
a plurality of appliances of claim 1, each contained in a water-impermeable envelope; and
a container for the plurality of appliance-containing envelopes.

10. An appliance adapted to transfer a composition from the interior surface of the appliance to the skin of a wearer of the appliance, the appliance comprising:
a substrate having a perimeter, the substrate comprising a water-impermeable layer sandwiched between, and attached to, an inner fibrous layer and an outer fibrous layer, and wherein the substrate is attached to itself in a way that defines an interior volume into which a wearer of the appliance may insert a portion of his or her body;
a composition deposited on at least a portion of the inner fibrous layer, wherein at least a portion of the composition is adapted to be transferred from the inner fibrous layer to the skin of the wearer of the appliance during use.

11. The appliance of claim 10 wherein the appliance is adapted to receive a foot of a user of the appliance.

12. The appliance of claim 10 wherein the composition is adapted to moisturize the skin of a user of the appliance.

13. The appliance of claim 12 wherein the composition comprises water and a humectant.

14. The appliance of claim 10 wherein the inner fibrous layer is a nonwoven.

15. The appliance of claim 10 wherein the inner fibrous layer, the outer fibrous layer, or both are attached to the water-impermeable layer at discrete locations.

16. The appliance of claim 15 wherein the inner fibrous layer, the outer fibrous layer, or both are gathered.

17. A package, the package comprising:
a plurality of appliances of claim 10, each contained in a water-impermeable envelope; and
a container for the plurality of appliance-containing envelopes.

18. A message adapted to be communicated to consumers, wherein the message is based, in whole or in part, on information relating to an appliance of claims 1 or 9, and wherein the message is contained in text, a symbol, a graphic, an image, and/or color, and wherein the message is embodied in a medium capable of being transmitted to consumers.
